(19) United States
(12) Patent Application Publication
(10) Pub. No.: US 2013/0183322 A1
(43) Pub. Date: Jul. 18, 2013

(54) IMMUNOSUPPRESSIVE DRUG COMBINATION FOR A STABLE AND LONG TERM ENGRAFTMENT

(71) Applicants: Yair Reisner, Old Jaffa (IL); Esther Bachar-Lustig, Rehovot (IL); Dalit Tchorsh-Yutsis, Rehovot (IL)

(72) Inventors: Yair Reisner, Old Jaffa (IL); Esther Bachar-Lustig, Rehovot (IL); Dalit Tchorsh-Yutsis, Rehovot (IL)

(73) Assignee: YEDA RESEARCH AND DEVELOPMENT CO., LTD., Rehovot (IL)

(21) Appl. No.: 13/821,269

(22) PCT Filed: Sep. 8, 2011
(86) PCT No.: PCT/IL11/00726

§ 371 Date: Mar. 7, 2013

(51) Int. Cl. A61K 39/395; A61K 35/26; A61K 35/28

(52) U.S. Cl. CPC A61K 39/3955; A61K 35/28; A61K 35/26
USPC 424/173.1; 424/184.1

(57) ABSTRACT

A method of treating a subject in need of a cell or tissue transplant is disclosed. The method comprising (a) transplanting a non-syngeneic cell or tissue transplant into the subject, wherein the transplant comprises bone marrow or lymphoid cells; and (b) administering to the subject a therapeutically effective amount of an immunosuppressive regimen comprising a Sphingosine 1-Phosphate Receptor Agonist, a B7 molecule inhibitor and a CD2/CD58 pathway inhibitor, thereby treating the subject.

### FIELD AND BACKGROUND OF THE INVENTION

The present invention, in some embodiments thereof, relates to an immunosuppressive drug combination and, more particularly, but not exclusively, to the use of same for inducing a stable and durable cell or tissue transplantation.

For many years, achieving specific and sustained immune tolerance has been the holy grail of transplantation medicine. One major approach to achieving this goal is by transplantation of hematopoietic stem cells that can potentially localize to the thymus, continuously present donor antigens, and thereby induce ongoing deletion of anti-donor T cell clones. This idea was presented more than 60 years ago and showed that intrauterine circulation exchange in cattle dizygotic twins achieved cross-tolerance to formed blood elements, however, achieving hematopoietic chimerism across major genetic barriers after birth was found over the years to present a difficult challenge.

Full donor type chimerism can be achieved even across major HLA disparity in patients receiving haploidentical transplants. The problem of graft versus host disease can be prevented by using extensively T cell depleted grafts, and the problem of graft rejection may be successfully overcome by using supralethal conditioning combined with megadoses of stem cells. However, while a high transplantation-related mortality rate of at least 20% (using HLA identical patients) might be reasonable in patients suffering from aggressive hematological malignancies, this rate is unacceptable for patients undergoing organ transplantation who are not under the threat of imminent death.

Recently, Kawai et al. [Kawai T. et al., N Engl J Med. (2008) 358:353-361] demonstrated in humans that several months following combined bone marrow (BM) and kidney transplants from HLA single-haplotype mismatched donors, all immunosuppressive therapy could be discontinued without significantly affecting transplant function. However, routine clinical application of this approach is limited by severe toxicity of the cytoreductive conditioning which is required in order to allow even transient engraftment of MHC-mismatched BM. Furthermore, although this approach required extensive immune depletion, only a short and transient hematopoietic chimerism was achieved. It is believed that the mechanism of tolerance in that system involves regulatory T cells inducing only transient peripheral tolerance.

To further improve the chimerism enabled by CD4+CD25+ T regulatory cells (Tregs), Pilat et al. transplanted whole mouse BM, including alloreactive T cells, under a co-stimulatory blockade with both anti-CD40L and CTLA4-Ig. Administration of host Tregs in conjunction with transient Rapamycin treatment resulted in low level chimerism without requiring any myeloablative pre-conditioning [Pilat N. et al., Am J Transplant. (2010) 10:1-12]. However, the use of anti-CD40L is problematic in humans due to its pro-thrombotic effect, and the requisite large number of Tregs used, might be difficult to collect from patients. Moreover, the inclusion of alloreactive T cells in the BM graft presents a risk for graft versus host disease (GVHD) which is unacceptable in applications involving non-malignant conditions.

In 1989 the present inventors demonstrated for the first time that rejection of allogeneic hematopoietic stem cell transplantation (HSCT) can be overcome by using large doses of hematopoietic stem cells [Lapidot T. et al., Blood (1989) 73:2025-2032]. However, a significant increase in stem cell inoculums has been difficult to achieve in humans. Using G-CSF to facilitate mobilization of hematopoietic CD34 stem cells from the BM and collecting these cells from peripheral blood significantly increased the number of progenitor cells that could be harvested from a single donor. Enriching conventional T cell depleted bone marrow (TDBM) with peripherally collected mobilized progenitor cells, made it possible to test the concept of stem cell dose escalation in humans. A pilot study conducted by Reisner Y. and Martelli M. F. showed for the first time that in humans, as in mice, cell dose escalation facilitated engraftment of T cell-depleted mismatched hematopoietic stem cell grafts [Aversa F et al. Blood (1994) 84:3948-3955; Reisner Y and Martelli Immunol Today (1995) 16:437-440]. After several modifications, an optimized protocol, using CD34+ cells isolated by Milteny magnetic beads, was developed and examined clinically in high-risk leukemia patients. Primary engraftment of haploidentical megadose transplants with low rates of GVHD was demonstrated in more than 93% of the patients and no GVHD prophylaxis was used [Aversa F et al., supra]. The few patients who failed to engraft achieved engraftment following a second transplant. Thus, by using megadoses of a purified stem cell graft, it is possible to overcome genetic barriers, using readily available haploidentical family members as a source for BM transplantation, and increasing the pool of stem cell donors especially for acute leukemia patients in remission. Subsequently, the present inventors showed that the mechanism by which CD34+ cells overcome the barrier presented by host T cells involves specific regulatory activity possessed by cells within the CD34+ cell fraction, inhibiting only host T cells directed against donor pMHC [Rachamim et al. Transplantation (1998) 65:1386-1393]. Furthermore this tolerizing activity was later shown, using limiting dilution analysis of alloreactive cytotoxic T cell precursors CTLp, to be mediated through a deletion based mechanism, by TNF-α induced apoptosis [Gur H et al. Blood. (2005) 105: 2585-2593]. Thus, inherent specificity, eliminating only host T cells directed against the donor Ags, while sparing other T cells that can further persist and fight infectious pathogens, could offer a specific and effective modality for the induction of transplantation tolerance.

Furthermore, the present inventors demonstrated that early hematopoietic progenitors cells within the Sca1+Lin⁻ cell fraction, are specifically able to reduce the frequency of anti-donor T cell clones both in vitro and in vivo, and induce mixed chimerism in sublethally irradiated recipient mice. This immune tolerance was also associated with specific tolerance toward donor-type skin grafts. However, primate studies suggested that further reduction of the conditioning to levels acceptable for organ transplantation requires stem cell numbers which cannot be realistically collected from human donors (Gan et al., unpublished results).

In previous studies attempting embryonic pancreas xeno-transplantation [Tchorsh-Yutsis D et al., Diabetes (2009) 58:1585-1594], the present inventors were able to achieve optimal maintenance of the embryonic graft upon transient treatment with anti-LFA-1 and anti-CD48 in conjunction with continuous immune suppression with FTY720. However, durable tolerance was not achieved and rejection ensued upon cessation of immune suppression. Likewise, engraftment was attained in 75% of mice transiently treated with anti-CD48 and CTLA4-Ig in conjunction with continuous FTY720 treatment. However, again, termination of FTY720 treatment led to graft rejection.

Additional background art includes U.S. Patent Application No. 20090041790, U.S. Patent Application No. 20100183612, U.S. Patent Application No. 20100166756, U.S. Patent Application No. 20100041602, U.S. Patent Application No. 20100022627, U.S. Patent Application No. 20100041602, U.S. Patent Application No. 20090068203, U.S. Patent Application No. 20090041790, U.S. Patent Application No. 20090041769, U.S. Patent Application No. 20090022730, U.S. Patent Application No. 20080160022, U.S. Patent Application No. 20070009511, U.S. Patent Application No. 20050214313, U.S. Patent Application No. 20050123539, U.S. Patent Application No. 20040022787, U.S. Patent Application No. 20030083246, U.S. Patent Application No. 20030022836 and U.S. Patent Application No. 20020182211.

### SUMMARY OF THE INVENTION

According to an aspect of some embodiments of the present invention there is provided a method of treating a subject in need of a cell or tissue transplant, the method comprising: (a) transplanting a non-syngeneic cell or tissue transplant into the subject, wherein the transplant comprises bone marrow or lymphoid cells; and (b) administering to the subject a therapeutically effective amount of an immunosuppressive regimen comprising a Sphingosine 1-Phosphate Receptor Agonist, a B7 molecule inhibitor and a CD2/CD58 pathway inhibitor, thereby treating the subject.

According to an aspect of some embodiments of the present invention there is provided a use of a Sphingosine 1-Phosphate Receptor Agonist, a B7 molecule inhibitor and a CD2/CD58 pathway inhibitor for reducing graft rejection of a non-syngeneic cell or tissue transplant in a subject, wherein the transplant comprises bone marrow or lymphoid cells.

According to some embodiments of the invention, the immunosuppressive regimen comprises a short term immunosuppressive regimen. According to some embodiments of the invention, the method further comprises conditioning the subject under sublethal, lethal or supralethal conditions prior to step (a).

According to some embodiments of the invention, the conditioning comprises non-myeloablative conditioning.

According to some embodiments of the invention, the conditioning comprises T cell debulking.

According to some embodiments of the invention, the T cell debulking comprises short term T cell debulking.

According to some embodiments of the invention, the conditioning comprises administration of an alkylating agent.

According to some embodiments of the invention, the alkylating agent comprises Busulphan.

According to some embodiments of the invention, the Sphingosine 1-Phosphate Receptor Agonist, the B7 molecule inhibitor and the CD2/CD58 pathway inhibitor are administered as part of a short term immunosuppressive regimen.

According to some embodiments of the invention, the bone marrow cells comprise T cell depleted bone marrow cells.

According to some embodiments of the invention, the bone marrow cells comprise hematopoietic precursor cells.

According to some embodiments of the invention, the cell or tissue transplant comprises a solid organ.

According to some embodiments of the invention, the Sphingosine 1-Phosphate Receptor Agonist is FTY720 and the B7 molecule inhibitor is a CTLA4-Ig and the CD2/CD58 pathway inhibitor is a soluble CD58-Ig.

According to some embodiments of the invention, the CD2/CD58 pathway inhibitor is selected from the group consisting of a soluble CD2 protein, a soluble CD58 protein, an anti-CD2 antibody and an anti-CD58 antibody.

According to some embodiments of the invention, the soluble CD58 protein comprises a soluble CD58-Ig.

According to some embodiments of the invention, the Sphingosine 1-Phosphate Receptor Agonist, the B7 molecule inhibitor and the CD2/CD58 pathway inhibitor are administered concomitantly.

According to some embodiments of the invention, the short term immunosuppressive regimen is effected for up to 6 months following transplantation.

According to some embodiments of the invention, administration of the Sphingosine 1-Phosphate Receptor Agonist is terminated 4 months following transplantation.

According to some embodiments of the invention, administration of the B7 molecule inhibitor and the CD2/CD58 pathway inhibitor is terminated 3 months following transplantation.

According to some embodiments of the invention, administration of the B7 molecule inhibitor and the CD2/CD58 pathway inhibitor is effected every two days following transplantation until day 6.

According to some embodiments of the invention, administration of the B7 molecule inhibitor and the CD2/CD58 pathway inhibitor is effected once a week from day 6 of transplantation until day 90.

According to some embodiments of the invention, the subject is a human subject.

According to some embodiments of the invention, the non-syngeneic cell or tissue transplant is derived from a donor selected from the group consisting of an HLA identical allogeneic donor, an HLA non-identical allogeneic donor and a xenogeneic donor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 demonstrates the chimerism induction protocol of the present invention utilizing non-myeloablative conditioning and co-stimulatory blockade. C3H/Hen recipient mice were conditioned with busulfan (2×30 mg/Kg) and T cell debulking with 300 mg anti-CD4 and anti-CD8. Post transplant treatment included 200 mg CTLA4/FC, 250 mg anti-CD48, and 0.1 mg FTY720 administered at the indicated time points.

FIGS. 2A-E are graphs demonstrating long term multilineage chimerism. FIG. 2A shows chimerism level 163 days after cessation of immune suppression; and FIGS. 2B-E show typical multilineage chimerism in the spleen of a chimeric mouse shown in FIG. 2A.

### DESCRIPTION OF SPECIFIC EMBODIMENTS OF THE INVENTION

The present invention, in some embodiments thereof, relates to an immunosuppressive drug combination and, more particularly, but not exclusively, to the use of same for inducing a stable and durable cell or tissue transplantation.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have uncovered that using a new combination of immunosuppressive drugs, namely a B7 molecule inhibitor (e.g. CTLA4-Ig), a CD2/CD58 pathway inhibitor (e.g. soluble CD58-Ig) and a Sphingosine 1-Phosphate Receptor Agonist (e.g. FTY720), leads to an efficient and durable engraftment of allogeneic T cell depleted bone marrow cells. Moreover, the present inventors have shown stable chimerism after cessation of immunosuppression with this novel immunosuppressive regimen.

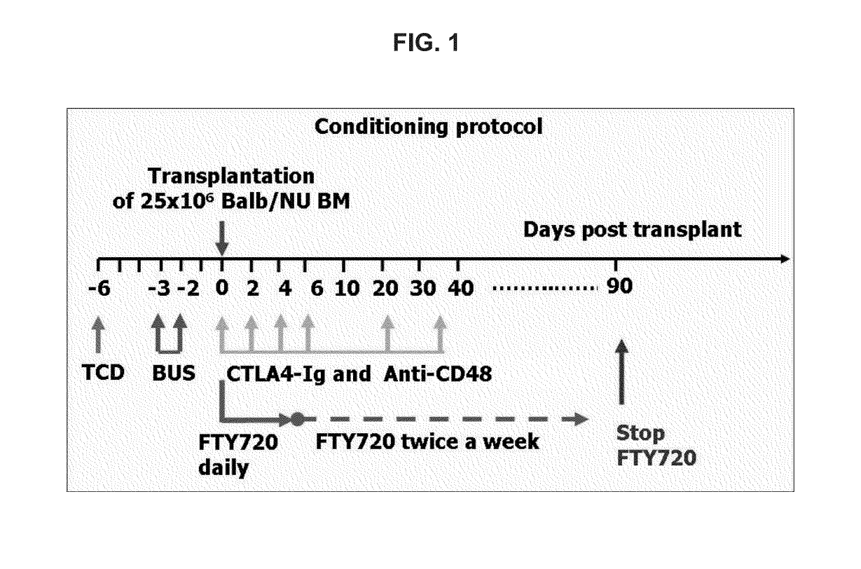

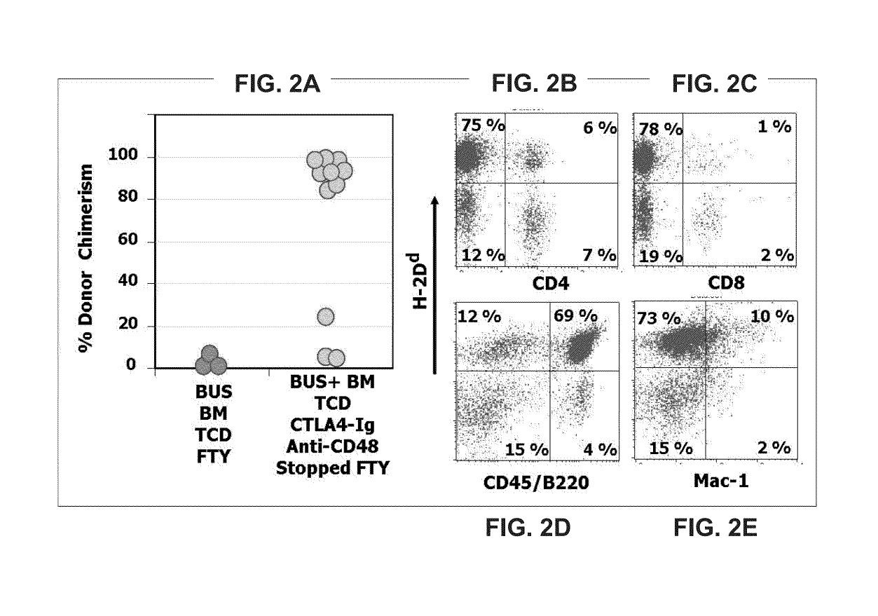

As is shown hereinbelow and in the Examples section which follows, the present inventors have established a stable chimerism in a mouse model by first conditioning the mice with minimal myeloablation (i.e. with busulfan and T cell debulking with anti-CD4 and anti-CD8, see FIG. 1). Next, the recipient mice were transplanted with allogeneic T cell depleted bone marrow cells (on day 0). Following transplantation, the mice were treated with a short term immunosuppressive regimen comprising CTLA4-Ig and anti-CD48 antibody (mouse CD48 is equivalent to human CD58) on days 0, 2, 4, 6, 21 and 35 and FTY720 daily on days 0 to 5 and twice a week from day 6 to day 90. Donor type chimerism was visible in recipient mice several months (2-5 months) after cessation of immune suppression (FIG. 2A). Furthermore, significant chimerism was attained in both the myeloid and lymphoid lineages (FIGS. 2B-E). Taken together, all these findings substantiate the combined use of a B7 molecule inhibitor (e.g. CTLA4-Ig), a CD2/CD58 pathway inhibitor (e.g. soluble CD58-Ig) and a Sphingosine 1-Phosphate Receptor Agonist (e.g. FTY720) for stable and long term engraftment.

Thus, according to one embodiment, there is provided a method of treating a subject in need of a cell or tissue transplant, the method comprising: (a) transplanting a non-syngeneic cell or tissue transplant into the subject, wherein the transplant comprises bone marrow or lymphoid cells; and (b) administering to the subject a therapeutically effective amount of an immunosuppressive regimen comprising a Sphingosine 1-Phosphate Receptor Agonist, a B7 molecule inhibitor and a CD2/CD58 pathway inhibitor, thereby treating the subject.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “subject” or “subject in need thereof” refers to a mammal, preferably a human being, male or female at any age that is in need of a cell or tissue transplantation. Typically the subject is in need of cell or tissue transplantation (also referred to herein as recipient) due to a disorder or a pathological or undesired condition, state, or syndrome, or a physical, morphological or physiological abnormality which is amenable to treatment via cell or tissue transplantation. Examples of such disorders are provided further below.

As used herein, the phrase “cell or tissue transplant” refers to a bodily cell (e.g. a single cell or a group of cells) or tissue (e.g. solid tissues or soft tissues, which may be transplanted in full or in part). Exemplary tissues which may be transplanted according to the present teachings include, but are not limited to, liver, pancreas, spleen, kidney, heart, lung, skin, intestine and lymphoid/hematopoietic tissues (e.g. lymph node, Peyer's patches thymus or bone marrow). Exemplary cells which may be transplanted according to the present teachings include, but are not limited to, hematopoietic stem cells (e.g. immature hematopoietic cells). Furthermore, the present invention also contemplates transplantation of whole organs, such as for example, kidney, heart, lung, liver or skin.

Depending on the application, the method may be effected using a cell or tissue which is non-syngeneic (i.e., allogeneic or xenogeneic) with the subject.

As used herein, the term “allogeneic” refers to a cell or tissue which is derived from a donor who is of the same species as the subject, but which is substantially non-clonal with the subject. Typically, outbred, non-zygotic twin mammals of the same species are allogeneic with each other. It will be appreciated that an allogeneic donor may be HLA identical or HLA non-identical with respect to the subject.

As used herein, the term “xenogeneic” refers to a cell or tissue which substantially expresses antigens of a different species relative to the species of a substantial proportion of the lymphocytes of the subject. Typically, outbred mammals of different species are xenogeneic with each other.

The present invention envisages that xenogeneic cells or tissues are derived from a variety of species such as, but not limited to, bovines (e.g., cow), equids (e.g., horse), porcines (e.g. pig), ovids (e.g., goat, sheep), felines (e.g., Felis domestica), canines (e.g., Canis domestica), rodents (e.g., mouse, rat, rabbit, guinea pig, gerbil, hamster) or primates (e.g., chimpanzee, rhesus monkey, macaque monkey, marmoset).

Cells or tissues of xenogeneic origin (e.g. porcine origin) are preferably obtained from a source which is known to be free of zoonoses, such as porcine endogenous retroviruses. Similarly, human-derived cells or tissues are preferably obtained from substantially pathogen-free sources.

According to an embodiment of the present invention, both the subject and the donor are humans.

Depending on the application and available sources, the cells or tissues of the present invention may be obtained from a prenatal organism, postnatal organism, an adult or a cadaver donor. Moreover, depending on the application needed the cells or tissues may be naive or genetically modified. Such determinations are well within the ability of one of ordinary skill in the art.

Any method know in the art may be employed to obtain a cell or tissue (e.g. for transplantation).

Transplanting the cell or tissue into the subject may be effected in numerous ways, depending on various parameters, such as, for example, the cell or tissue type; the type, stage or severity of the recipient's disease (e.g. organ failure); the physical or physiological parameters specific to the subject; and/or the desired therapeutic outcome.

Transplanting a cell or tissue transplant of the present invention may be effected by transplanting the cell or tissue transplant into any one of various anatomical locations, depending on the application. The cell or tissue transplant may be transplanted into a homotopic anatomical location (a normal anatomical location for the transplant), or into an ectopic anatomical location (an abnormal anatomical location for the transplant). Depending on the application, the cell or tissue transplant may be advantageously implanted under the renal capsule, or into the kidney, the testicular fat, the sub cutis, the omentum, the portal vein, the liver, the spleen, the heart cavity, the heart, the chest cavity, the lung, the skin, the pancreas and/or the intra abdominal space.

For example, a liver tissue according to the present teachings may be transplanted into the liver, the portal vein, the renal capsule, the sub-cutis, the omentum, the spleen, and the intra-abdominal space. Transplantation of a liver into various anatomical locations such as these is commonly practiced in the art to treat diseases amenable to treatment via hepatic transplantation (e.g. hepatic failure). Similarly, transplanting a pancreatic tissue according to the present invention may be advantageously effected by transplanting the tissue into the portal vein, the liver, the pancreas, the testicular fat, the sub-cutis, the omentum, an intestinal loop (the subserosa of a U loop of the small intestine) and/or the intra-abdominal space. Transplantation of pancreatic tissue may be used to treat diseases amenable to treatment via pancreatic transplantation (e.g. diabetes). Likewise, transplantation of tissues such as a kidney, a heart, a lung or skin tissue may be carried out into any anatomical location described above for the purpose of treating recipients suffering from, for example, renal failure, heart failure, lung failure or skin damage (e.g., bums).

The method of the present invention may also be used, for example, for treating a recipient suffering from a disease requiring hematopoietic stem cell transplantation (e.g. immature hematopoietic cells). Such a disease includes, but is not limited to, leukemia such as acute lymphoblastic leukemia (ALL), chronic lymphocytic leukemia (CLL)/small lymphocytic lymphoma, acute non-lymphoblastic leukemia (ANLL), acute myelocytic leukemia (AML), chronic myelocytic leukemia (CML), hairy cell leukemia, T-cell prolymphocytic leukemia, B-cell prolymphocytic leukemia and Juvenile myelomonocytic leukemia; lymphoma such as Hodgkin lymphoma, Burkitt's lymphoma, diffuse large B-cell lymphoma (DLBCL), precursor T-cell leukemia/lymphoma, follicular lymphoma, mantle cell lymphoma, MALT lymphoma, B-cell chronic lymphocytic leukemia/lymphoma and Mycosis fungoides; severe combined immunodeficiency syndromes (SCID), including adenosine deaminase (ADA), osteopetrosis, aplastic anemia, Gaucher's disease, thalassemia and other congenital or genetically-determined hematopoietic abnormalities.

Immature allogeneic or xenogeneic hematopoietic cells (including stem cells) which can be derived, for example, from bone marrow, mobilized peripheral blood (by for example leukapheresis), fetal liver, yolk sac and/or cord blood of the donor and which are typically T-cell depleted CD34+ immature hematopoietic cells, can be transplanted to a recipient suffering from a disease.

According to a specific embodiment of the present invention, the transplant comprises bone marrow or lymphoid cells. According to another embodiment of the present invention, the cell transplant comprises T cell depleted bone marrow cells. According to another embodiment of the present invention, the cell transplant comprises hematopoietic precursor cells.

Thus, the subject may be administered with a dose of cells ranging from about 10×10⁶ to about 10×10⁹ cells per kg.

It will be appreciated that the immature allogeneic or xenogeneic hematopoietic cells of the present invention may be transplanted into a recipient using any method known in the art for cell transplantation, such as but not limited to, cell infusion (e.g. I.V.), via an intraperitoneal route or via intrabone route.

Optionally, when transplanting a cell or tissue transplant of the present invention into a subject having a defective organ, it may be advantageous to first at least partially remove the failed organ from the subject so as to enable optimal development of the transplant, and structural/functional integration thereof with the anatomy/physiology of the subject.

The method of the present invention also envisions co-transplantation of several organs (e.g. heart and bone marrow e.g. hematopoietic stem cells, kidney and bone marrow e.g. hematopoietic stem cells, etc.) in case the subject may be beneficially effected by such a procedure.

Following transplantation of the cell or tissue transplant into the subject according to the present teachings, it is advisable, according to standard medical practice, to monitor the growth functionality and immuno-compatibility of the organ according to any one of various standard art techniques. For example, the functionality of a pancreatic tissue transplant may be monitored following transplantation by standard pancreas function tests (e.g. analysis of serum levels of insulin). Likewise, a liver tissue transplant may be monitored following transplantation by standard liver function tests (e.g. analysis of serum levels of albumin, total protein, ALT, AST, and bilirubin, and analysis of blood-clotting time). Structural development of the cells or tissues may be monitored via computerized tomography, or ultrasound imaging.

Regardless of the transplant type, in order to reduce, by at least about 30%, 40%, 50%, 60%, 70%, 80%, 90% or 95%, or preferably avoid graft rejection, the present invention contemplates administration of an immunosuppressive regimen comprising a Sphingosine 1-Phosphate Receptor Agonist, a B7 molecule inhibitor and a CD2/CD58 pathway inhibitor.

As used herein, the term “Sphingosine 1-Phosphate Receptor Agonist” refers to a molecule which activates signaling through the Sphingosine 1-Phosphate Receptor. Typically, this molecule acts as a superagonist of the Sphingosine 1-Phosphate Receptor (e.g. on thymocytes and lymphocytes) and induces aberrant internalization of the receptor and sequestering of the lymphocytes in the lymph nodes. Thus, determining activation of the Sphingosine 1-Phosphate Receptor Agonist may be carried out for example by peripheral lymphocyte counts (i.e. reduction thereof). In a specific embodiment, the Sphingosine 1-Phosphate Receptor Agonist refers to the synthetic compound 2-amino-2-[2-(4-octylphenyl)ethyl]propane-1,3-diol hydrochloride also named Fingolimod or FTY720. Sphingosine 1-Phosphate Receptor Agonist is commercially available from e.g. Novartis (Gilenia®). Examples of FTY720 analogues, include but are not limited to, (S)-phosphonate analog of FTY720.

As used herein, the term “B7 molecule inhibitor” refers to a molecule which specifically binds and inhibits activation of the B7 molecules e.g. B7.1 (CD80) and B7.2 (CD86). In a specific embodiment, the B7 molecule inhibitor is a soluble CTLA4 protein, for example a CTLA4 fusion protein, such as with an immunoglobulin domain which confers serum stability (e.g., CTLA4-Ig).

As used herein, the term “CTLA4-Ig” refers to a human fusion protein with immunosuppressive activity. It consists of the binding domain of human cytotoxic T-lymphocyte-associated antigen 4 and human IgG1. CTLA4-Ig works by binding to CD80 and CD86 (i.e. B7.1 and B7.2, respectively) on antigen presenting cells, thereby blocking the engagement of CD28 on T-cells, a co-stimulatory signal required for full T-cell activation. This co-stimulatory blocker prevents T-cell activation, proliferation, and subsequent cytokine production. This T-cell regulatory protein may be useful in treating autoimmune diseases such as rheumatoid arthritis, and may help prevent organ transplant rejection. CTLA4-Ig is commercially available from e.g. Bristol-Myers Squibb as Abatacept (marketed as Orencia) and as Belatacept.

As used herein, the term “CD2/CD58 pathway inhibitor” refers to a molecule which specifically binds and blocks the co-stimulatory CD58/CD2 interaction. The CD2/CD58 pathway inhibitor may comprise a soluble CD2 protein, a soluble CD58 protein [i.e. soluble leukocyte function antigen-3 (LFA-3) protein], an anti-CD2 antibody or an anti-CD58 antibody (i.e. anti-LFA-3 antibody). Thus, for example, the soluble CD58 protein may comprise a CD58 fusion protein comprising the extracellular CD2-binding portion of CD58/LFA-3 fused with an immunoglobulin domain (hinge, CH2 and CH3 domains) portion of human IgG1 which confers serum stability (e.g., soluble CD58-Ig). Such a soluble CD58-Ig fusion protein includes, but is not limited to, Alefacept (brand name Amevive). According to another specific embodiment, the CD2/CD58 pathway inhibitor comprises an antibody such as a monoclonal anti-CD58/LFA-3 antibody [commercially available from e.g. Millipore (CHEMICON/Upstate/Linco) e.g. clone bric 5] or an anti-CD2 antibody (commercially available from e.g. Abcam e.g. Clone MEM-65).

Methods of generating antibodies and Ig fusion proteins are well known in the art.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference). Methods of humanizing antibodies are available from Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

The immunosuppressive regimen of the present invention may be administered to the subject prior to, concomitantly with, or following transplantation of the cell or tissue transplant.

Thus, for example, and as taught in the Examples section which follows, the B7 molecule inhibitor (e.g. CTLA4-Ig) and/or CD2/CD58 pathway inhibitor (e.g. soluble CD58-Ig) may be administered to the subject beginning on the day of transplantation (i.e. day 0) and continuously every two days until day 6. Then, the B7 molecule inhibitor (e.g. CTLA4-Ig) and/or CD2/CD58 pathway inhibitor may be administered every two weeks from day 6 until day 35 of transplantation. Sphingosine 1-Phosphate Receptor Agonist (e.g. FTY720) may be administered to the subject daily from days 0 to 5 of transplantation and twice a week from day 6 until day 90 of transplantation.

According to a specific embodiment of the present invention, the immunosuppressive regimen is administered to the subject for a short term.

As used herein, the phrase “short term” refers to a transient treatment, i.e. not a chronic treatment. According to an embodiment of the present invention, the immunosuppressive regimen is administered to the subject for less than a year, less than 10 months, less than 8 months, less than 6 months, less than 5 months, less than 4 months or less than 3 months after transplantation.

Treatment may be initiated as daily treatment, followed by bi-weekly administration, weekly administration, once in every two weeks, once a month etc. The subject is monitored for graft rejection as described above.

According to a specific embodiment of the present invention, administration of a B7 molecule inhibitor (e.g. CTLA4-Ig) and/or a CD2/CD58 pathway inhibitor may be terminated 20 days, 25 days, 30 days, 35 days, 40 days, 45 days, 50 days, 55 days, 60 days, 65 days, 70 days, 75 days, 80 days, 85 days, 90 days, 100 days, 110 days, 120 days, 5 months, 6 months, 7 months, 8 months, 9 months, 10 months, 11 months, 12 months, 18 months or 24 months following transplantation. Likewise, administration of Sphingosine 1-Phosphate Receptor Agonist (e.g. FTY720) may be terminated 50 days, 55 days, 60 days, 65 days, 70 days, 75 days, 80 days, 85 days, 90 days, 95 days, 100 days, 105 days, 110 days, 115 days, 120 days, 5 months, 6 months, 7 months, 8 months, 9 months, 10 months, 11 months, 12 months, 18 months or 24 months following transplantation.

It will be appreciated that the B7 molecule inhibitor (e.g. CTLA4-Ig), CD2/CD58 pathway inhibitor and Sphingosine 1-Phosphate Receptor Agonist (e.g. FTY720) may be administered to the subject concomitantly or subsequent to each other over the course of treatment.

Without being bound to theory, a therapeutically effective amount is an amount of immunosuppressive regimen efficient for reducing graft rejection in a subject. Since the immunosuppressive regimen of the present invention may be administered to the subject for a short term, higher doses of B7 molecule inhibitor (e.g. CTLA4-Ig), CD2/CD58 pathway inhibitor and Sphingosine 1-Phosphate Receptor Agonist (e.g. FTY720) may be needed to achieve the beneficial effects of the regimen (e.g. reducing graft rejection).

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

For example, in case of T cell depleted bone marrow transplantation, the dose of Sphingosine 1-Phosphate Receptor Agonist (e.g. FTY720) administered to the subject starting from about one week before transplantation until about 5 weeks post transplantation should range from about 0.5 mg/kg to about 1.5 mg/kg, about 0.75 mg/kg to about 1.25 mg/kg or about 1 mg/kg. The dose of Sphingosine 1-Phosphate Receptor Agonist (e.g. FTY720) administered to the subject starting from about week five post-transplantation until about 120 days post-transplantation should range from about 0.1 mg/kg to about 1.0 mg/kg, about 0.2 mg/kg to about 0.6 mg/kg or about 0.3 mg/kg. According to a specific embodiment, the dose of Sphingosine 1-Phosphate Receptor Agonist (e.g. FTY720) is administered daily.

For example, in case of T cell depleted bone marrow transplantation, the dose of B7 molecule inhibitor (e.g. CTLA4-Ig such as Belatacept) administered to the subject should range from about 0.5 mg/kg to about 50 mg/kg, about 1.0 mg/kg to about 40 mg/kg, about 2.0 mg/kg to about 30 mg/kg or about 20 mg/kg. According to a specific embodiment, (e.g. CTLA4-Ig such as Belatacept) is administered on days 0, 4 and 7 of transplantation, followed by once weekly until about day 60 post-transplantation.

For example, in case of T cell depleted bone marrow transplantation, the dose of CD2/CD58 pathway inhibitor (e.g. Alefacept) administered to the subject should range from about 0.1 mg/kg to about 1.0 mg/kg, about 0.2 mg/kg to about 0.6 mg/kg or about 0.6 mg/kg. According to a specific embodiment, LFA-3/CD58 inhibitor (e.g. Alefacept) is administered intramuscularly (I.M.) on days 0, 4, and 7 of transplantation, followed by once weekly administrations until about day 60 post-transplantation.

The number of administrations, the duration of administrations and the therapeutically effective amount of the immunosuppressive regimen described herein may be adjusted as needed taking into account the type of transplantation and the subject's response to the regimen. Determination of the number of administrations, the duration of administrations and the therapeutically effective amount is well within the capability of those skilled in the art, especially in light of the detailed disclosure provided herein.

In order to facilitate engraftment of the cell or tissue transplant, the method may further advantageously comprise conditioning the subject with an additional immunosuppressive drug and/or immunosuppressive irradiation prior to, concomitantly with or following transplantation of the cell or tissue transplant.

Thus, according to an embodiment of the present invention, the subject is conditioned under sublethal, lethal or supralethal conditions prior to transplantation of a cell or tissue transplant.

Thus, for example, the subject may be treated with a myeloablative or non-myeloablative conditioning. Such conditioning may comprise, for example and as described in detail in the Examples section which follows, T cell debulking e.g. by anti-CD4 antibody and anti-CD8 antibody or with anti-thymocyte globulin (ATG) (e.g. 6 days prior to transplantation) and treatment with an alkylating agent such as Busulfan, Myleran or Busulfex (e.g. 3 and 2 days prior to transplantation, e.g. at a dose of about 8 mg per kg). According to a specific embodiment of the present invention, T cell debulking is effected for a short term.

Ample guidance for selecting and administering suitable immunosuppressive agents for transplantation is provided in the literature of the art (for example, refer to: Kirkpatrick C H. and Rowlands D T Jr., 1992. JAMA. 268, 2952; Higgins R M. et al., 1996. Lancet 348, 1208; Suthanthiran M. and Strom T B., 1996. New Engl. J. Med. 331, 365; Midthun D E. et al., 1997. Mayo Clin Proc. 72, 175; Morrison V A. et al., 1994. Am J Med. 97, 14; Hanto D W., 1995. Annu Rev Med. 46, 381; Senderowicz A M. et al., 1997. Ann Intern Med. 126, 882; Vincenti F. et al., 1998. New Engl. J. Med. 338, 161; Dantal J. et al. 1998. Lancet 351, 623).

Suitable routes of administration of the immunosuppressive regimen of the present teachings may include, for example, oral, rectal, transmucosal, especially transnasal, intestinal or parenteral delivery, including intramuscular, subcutaneous and intramedullary injections as well as intrathecal, direct intraventricular, intracardiac, into the common coronary artery, intravenous, intraperitoneal, intranasal, or intraocular injections.

The immunosuppressive agents of the present invention may be packed in an article of manufacture comprising at least one packaging material packaging an immunosuppressive agent. In a specific embodiment, the package comprises all three agents i.e., B7 molecule inhibitor (e.g. CTLA4-Ig), CD2/CD58 pathway inhibitor and Sphingosine 1-Phosphate Receptor Agonist (e.g. FTY720). In another specific embodiment, Sphingosine 1-Phosphate Receptor Agonist (e.g. FTY720) is packaged in a separate package while the B7 molecule inhibitor (e.g. CTLA4-Ig) and CD2/CD58 pathway inhibitor are co-formulated. In another specific embodiment, each of the immunosuppressive agents i.e. Sphingosine 1-Phosphate Receptor Agonist (e.g. FTY720), B7 molecule inhibitor (e.g. CTLA4-Ig) and CD2/CD58 pathway inhibitor is packaged in a separate package. The article of manufacture may comprise instructions for use in the treatment of a subject undergoing a cell or tissue transplant (in line with the guidelelines provided above).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Experimental Procedures

Animals: 6-12 week old female mice were used of the following strains: C57BL/6 (B6, recipient, H-2b, Ly-5.2), B6.SJL-Ptprca Pep3b/BoyJ (congenic strain, donor, H-2b, Ly-5.1) Balb/c (donor, H-2d) and C3H/Hen (recipient, H-2k) mice (all purchased from Harlan Laboratories Ltd, Ein Kerem Breeding farm Jerusalem). All mice were housed under specific pathogen free conditions and maintained under conditions approved by the Institutional Animal Care and Use Committee at the Weizmann Institute of Science.

Determination of the minimal myeloablation with Busulphan: Different groups of C57BL/6 (Ly-5.2) recipient mice were conditioned with the following final doses of IV Busulfex (Otsuka America Pharmaceutical, Inc.): 10, 20, 40, 50, 60, 80, and 100 mg/Kg/mouse. The final doses of IV Busulfex were divided in two and administered (IP) on day −2 and −1. On day 0, 25×10⁶ T-depleted bone marrow cells isolated from B6.SJL-Ptprca Pep3b/BoyJ (Ly-5.1) donors were transplanted (IV) and the establishment of donor type chimerism was defined 1 and 12 month post transplant in the blood, spleen, and BM by FACS analysis of the congenic donor marker (Ly-5.1). Phenotypic expression analysis of CD8, CD4, CD45/B220 and CD11b markers on the LY-5.1⁺ (donor) cells in the spleen and the BM of the donor chimera, was performed 12 month post transplant.

Non myeloablative conditioning and co-stimulatory blockade Protocol: C3H/Hej (H-2K^{k}) recipient mice were conditioned with 30 mg/kg IV Busulfex on days −3 and −2 following T cell debulking on day −6 with 300 μg anti-CD4 (Bio Express, clone Gk1.5) and anti-CD8 (Bio Express, cone 53.6.72) antibodies. On day 0, 25×10⁶ T-depleted BM cells from Balb/c-Nude (H-2D^{d}) donors were transplanted and subjected to co-stimulation blockade consisting of 200 μg CTLA4/FC (Chimerigen Laboratories), 250 μg anti-CD48 (Bio Express, clone HM 48) and 0.1 mg FTY720 (Novartis) that was administered as follows: CTLA4/FC and anti-CD48 were injected IP on days 0, 2, 4, 6, 21 and 35 while FTY720 was inoculated per OS for 5 days from day 0 to 5 and from day 6 till day 90 twice a week. Chimerism analysis was monitored every 30 days by FACS analysis.

Flow cytometry for chimerism and multilineage analysis: For chimerism analysis, blood mononuclear cells were stained with labeled antibodies specific for Host (H-2K^{k}-phycoerythrin (PE)) and donor (H-2D^{d}-fluorescein isothiocyanate (FITC)) MHC class-I antigens. In the congenic model, anti-CD45.2-PE and anti-CD45.1-FITC antibodies were used to distinguish between the host and the donor.

Multilineage Chimerism was performed on donor chimera 70 to 163 days post transplant. Splenocytes were multi-color stained with antibodies against Host (H-2K^{k}-PE), donor (H-2D^{d}-FITC) and the following lineage markers: Anti-CD4-Allophycocyanin (APC), Anti-CD8-APC, Anti-CD45/B220-PE and CD11b-PE. All staining were performed according to the manufacturer instructions (BD-Pharmingen). Fluorescence-activated cell sorting (FACS) analysis was performed using a modified Becton Dickinson FACScan.

### Example 1

### Establishment of a Mouse Model for Minimal Conditioning

Considering the importance of providing empty niches for successful BM engraftment, the present inventors initially determined the minimal myeloablation with busulphan which induced durable chimerism following infusion of congenic B6-SJL (Ly-5.1) T cell depleted bone marrow (TDBM, 25×10⁶) into B6 (Ly-5.2) mice. Testing doses ranging from 10 mg/Kg to 100 mg/Kg busulphan, the present inventors showed that donor type chimerism above 50% was attained at doses higher than 50 mg/Kg (40±26%, 66±7% and 75±2% chimerism at 50, 60, and 100 mg/Kg, respectively). Consequently, the sublethal dose of 60 mg/Kg was selected for further use in all attempts to induce allogeneic chimerism, in conjunction with transient debulking of host lymphocytes by a single infusion of anti-CD4 and anti-CD8 depleting antibodies.

### Example 2

### Chimerism Induction with New Clinically Feasible Agents

The well tolerated combined sublethal conditioning described in Example 1 above presented a formidable barrier for engraftment of allogeneic ‘megadose’ T cell depleted bone marrow, and no chimerism was achieved when using bone marrow (BM) alone or BM with FTY720.

However, addition of transient post transplant treatment with CTLA4-Ig, anti-CD48 and FTY720 (FIG. 1) led, in two independent experiments, to marked donor type chimerism with a median follow-up of 116 days (range of 70 to 163 days) beyond cessation of immune suppression (FIG. 2A). Thus, while no chimerism could be detected in mice treated post transplant with FTY alone (0 out of 7 mice), transient post transplant immune suppression with CTLA4-Ig, anti CD48 and FTY720 resulted in more than 80% donor type chimerism in 8 of 11 mice. As can be seen in FIGS. 2B-E, significant chimerism was attained in both the myeloid and lymphoid lineages.

Since agents such as Belatacept (CTLA4-Ig) and Alefacept (blocking the interaction of CD48) are available for clinical use, the present results suggest a potentially feasible co-stimulatory blockade approach for the induction of durable hematopoietic chimerism under non-myeloablative conditioning, as a platform for cell therapy and organ transplantation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating a subject in need of a cell or tissue transplant, the method comprising:
(a) transplanting a non-syngeneic cell or tissue transplant into the subject, wherein said transplant comprises bone marrow or lymphoid cells; and
(b) administering to the subject a therapeutically effective amount of an immunosuppressive regimen comprising a Sphingosine 1-Phosphate Receptor Agonist, a B7 molecule inhibitor and a CD2/CD58 pathway inhibitor, thereby treating the subject.

2. The method of claim 1, wherein said immunosuppressive regimen comprises a short term immunosuppressive regimen.

3. The method of claim 1, further comprising conditioning the subject under sublethal, lethal or supralethal conditions prior to step (a).

4. The method of claim 3, wherein said conditioning comprises non-myeloablative conditioning.

5. The method of claim 3, wherein said conditioning comprises T cell debulking.

6. The method of claim 5, wherein said T cell debulking comprises short term T cell debulking.

7. The method of claim 3, wherein said conditioning comprises administration of an alkylating agent.

8. The method of claim 7, wherein said alkylating agent comprises Busulphan.

9-10. (canceled)

11. The method of claim 1, wherein said bone marrow cells comprise T cell depleted bone marrow cells.

12. The method of claim 11, wherein said bone marrow cells comprise hematopoietic precursor cells.

13. The method of claim 1, wherein said cell or tissue transplant comprises a solid organ.

14. The method of claim 1, wherein said Sphingosine 1-Phosphate Receptor Agonist is FTY720 and said B7 molecule inhibitor is a CTLA4-Ig and said CD2/CD58 pathway inhibitor is a soluble CD58-Ig.

15. The method of claim 1, wherein said CD2/CD58 pathway inhibitor is selected from the group consisting of a soluble CD2 protein, a soluble CD58 protein, an anti-CD2 antibody and an anti-CD58 antibody.

16. The method of claim 15, wherein said soluble CD58 protein comprises a soluble CD58-Ig.

17. The method of claim 1, wherein said Sphingosine 1-Phosphate Receptor Agonist, said B7 molecule inhibitor and said CD2/CD58 pathway inhibitor are administered concomitantly.

18. The method of claim 2, wherein said short term immunosuppressive regimen is effected for up to 6 months following transplantation.

19. The method of claim 18, wherein administration of said Sphingosine 1-Phosphate Receptor Agonist is terminated 4 months following transplantation.

20. The method of claim 18, wherein administration of said B7 molecule inhibitor and said CD2/CD58 pathway inhibitor is terminated 3 months following transplantation.

21. The method of claim 18, wherein said administration of said B7 molecule inhibitor and said CD2/CD58 pathway inhibitor is effected every two days following transplantation until day 6.

22. The method of claim 21, wherein said administration of said B7 molecule inhibitor and said CD2/CD58 pathway inhibitor is effected once a week from day 6 of transplantation until day 90.

23. The method of claim 1, wherein said subject is a human subject.

24. The method of claim 1, wherein said non-syngeneic cell or tissue transplant is derived from a donor selected from the group consisting of an HLA identical allogeneic donor, an HLA non-identical allogeneic donor and a xenogeneic donor.
